SEQUENCE LISTING (4 sequences)

SEQ ID NO: 1 - DNA, 59 bp, Artificial sequence
  Primer
acccatggggacacaccagcccgaggagcgccccgaatgattccggggatccgtcgacc

SEQ ID NO: 2 - DNA, 58 bp, Artificial sequence
  Primer
gctgtcagaggtgcggatcggggatgggcggtgcggtcatgtaggctggagctgcttc

SEQ ID NO: 3 - DNA, 20 bp, Artificial sequence
  Primer
cccgaggagcgccccgaatg

SEQ ID NO: 4 - DNA, 20 bp, Artificial sequence
  Primer
ggggatgggcggtgcggtca

(19) United States
(12) Patent Application Publication
(10) Pub. No.: US 2007/0059704 A1
(43) Pub. Date: Mar. 15, 2007

(54) Detection system

(75) Inventors: Matthew Ian Hutchings, NORWICH (GB); Hee-Jeon Hong, Norwich (GB); Mark Jeremy Buttner, Norwich (GB)

Correspondence Address: MICHAEL BEST & FRIEDRICH, LLP, ONE SOUTH PINCKNEY STREET, P O BOX 1806, MADISON, WI, 53701, US

(73) Assignee: PLANT BIOSCIENCE LIMITED, NORWICH (GB)

(21) Appl. No.: 10/562,247

(22) PCT Filed: Jun. 23, 2004
(86) PCT No.: PCT/GB04/02685

§ 371 Date: Sep. 13, 2006

(30) Foreign Application Priority Data
Jun. 24, 2003 (GB) 0314699.0

(51) Int. Cl. C12Q 1/68

(52) U.S. Cl. 435/6

(57) ABSTRACT

The invention provides a methods and materials for detecting an activity of a glycopeptide antibiotic (such as a vancomycin-type antibiotic), in a sample, the method comprising the steps of: (a) providing a microorganism in which a first endogenous gene encoding peptidyltransferase activity is inactivated, which activity is necessary for growth of the microorganism, and which activity can be complemented by a second, different, peptidyltransferase, which second peptidyltransferase is inducible in the microorganism by the presence of the antibiotic, (b) contacting the sample with the microorganism, (c) observing the microorganism for growth, wherein growth of the microorganism is correlated with the presence of the antibiotic. Unlike systems of the prior art, the system does not rely on the expression of a heterologous reporter gene in order to detect antibiotic activity, but instead utilizes a “drug dependent” microorganism that can only grow in the presence of antibiotics that act an inducers of the relevant genes.

### TECHNICAL FIELD

The present invention relates to methods and materials for screening for compounds which have antibiotic activity. It further relates to methods for generating microorganisms having utility in screening, tools which can be generally used in such methods, the microorganisms themselves, and detection methods employing the microorganisms.

### BACKGROUND ART

There is an ongoing requirement for novel compounds that have antibiotic activity, for instance to counteract the problem of drug resistance. Methods for screening potential sources of antibiotic that have been used in the prior art include those which are based on particular ‘indicator’ or ‘reporter’ strains of bacteria.

A vanHp-cat fusion (regulated by VanS/VanR—see Ulijasz et al. (1996) was used by Lai and Kirsch (1996) to assay more than 6,800 compound for induction of vanHp in Enterococcus faecium.

WO 01/92559 (Plant Bioscience Limited) discloses a system in which heterologous reporter genes are used to detect the induction by antibiotics of multi-component cell wall responsive signal transduction systems. This is exemplified by inter alia CseB\C (regulates the sigE promoter), and Van S\R (regulates the vanH promoter).

WO 03/012128 (Plant Bioscience Limited) discloses a Streptomyces coelicolor based system in which heterologous reporter genes are used to detect the induction by antibiotics of a promoter from the vanSREFHAX cluster.

### DISCLOSURE OF THE INVENTION

The present inventors have devised a novel system to provide a screen for cell wall-specific antibiotics. Unlike systems of the prior art, this does not rely on the expression of a heterologous reporter gene in order to detect antibiotic activity. In embodiments of the invention, the inventors provide a “drug dependent” bacterial mutant strain that can only grow in the presence of glycopeptides (such as vancomycin) that act as inducers of the van genes. Thus the presence of such antibiotics is readily and robustly detected by viability of the strain.

As a test system, the inventors have demonstrated that the activation of vanF and vanHAX genes of Streptomyces coelicolor can compensate for the loss of activity in femX null mutants (note the equivalent gene designations in the S. coelicolor genome sequence (www.sanger.ac.uk/Projects/S_{—}coelicolor/) are as follows: vanF=SC03593; vanH=SC03594; vanA=SC03595; vanX=SC03596; femX=SC03904).

Briefly, although not wishing to be bound by any particular mechanism or underlying specificity, it is understood that when expressed in active form, FemX adds a single cross-bridge glycine to the stem pentapeptide of S. coelicolor cell wall precursors (FIG. 1). Because transpeptidation of this cross-bridge (FIG. 2) is essential for mature cell wall formation, FemX is an essential protein under ‘normal’ conditions. However, through the action of VanHAX enzymes, induction of the van genes remodels cell wall precursors such that the stem pentapeptide terminates D-ala-D-lac instead of D-ala-D-ala (not shown) and this can also be subject to transpeptidation (FIG. 3). S. coelicolor FemX cannot recognize precursors terminating D-ala-D-lac; instead the van cluster encodes a FemX homologue, VanF, which can recognize precursors terminating D-ala-D-lac as a substrate, and is therefore able to add the single glycine cross-bridge (FIG. 1).

Irrespective of the precise specificity, it can be seen from the disclosure herein that as a consequence, femX is non-essential provided that vanF is expressed, and femX null mutants are viable in the presence of vancomycin or other inducers of the van genes, but die in the absence of such inducers (FIG. 4). This creates a drug-dependent strain that can only grow in the presence of van gene inducers (FIG. 4).

Thus in a first aspect of the invention there is disclosed a method of detecting an activity of a glycopeptide, such as an antibiotic, in a sample, the method comprising the steps of:
(a) providing a microorganism in which a first endogenous gene encoding peptidyltransferase activity is inactivated, which activity is necessary for growth of the microorganism, and which activity can be complemented by a second, different, peptidyltransferase, which second peptidyltransferase is inducible in the microorganism by the presence of the antibiotic,
(b) contacting the sample with the microorganism,
(c) observing the microorganism for growth,
   wherein growth of the microorganism is correlated with the presence of the antibiotic.

The term “antibiotic” is used broadly in this aspect to include glycopeptidic compounds (natural, semi-synthetic or synthetic), which have the potential to inhibit or kill (susceptible) microorganisms generally by interfering with the physical integrity of the cell envelope.

The precise specificity of the screen will depend on the specificity of induction of the second peptidyltransferase. In preferred embodiments of the invention (such as those based on inactivation of FemX and the induction and activity of VanF), it is particularly effective for the detection of glycopeptide compounds such as cell wall-specific antibiotics that have the potential to induce the van genes—these include ristocetin and vancomycin.

By “observing” is meant ascertaining by any means (directly or indirectly) the growth or failure of growth of the microorganism.

The activity detected may be correlated with the presence or absence of an antibiotic, or putative antibiotic, in the sample in a qualitative manner. Alternatively it may be used to make a quantitative assessment.

Some particular embodiments and aspects will now be discussed in more detail.

Samples may be selected from any suitable source. In particular, samples may be selected from culture supernatants and extracts from soil isolates, compounds produced by chemical synthesis including combinatorial chemistry; and compounds produced by combinatorial biosynthesis.

The assay may use any suitable species of bacteria. Preferably the assay uses an actinomycete such as a strain of Streptomyces e.g. S. coelicolor or S. avermetilis.

One preferred bacterium is M600, which is a plasmid-free derivative of wild-type S. coelicolor A3(2).

In addition, it may be preferred to use strains in which enzymes which may otherwise degrade glycopeptidic antibiotics (thereby reducing the sensitivity of the assay) have been inactivated.

In preferred embodiments the second peptidyltransferase will also be endogenous, although if it is not present, and if desired, the gene encoding it (and any ancillary enzymes preferred or required) could be introduced into it.

In preferred embodiments the peptidyltransferase activity is nonribosomal and operates on a substrate in the cell involved in cross-bridge formation of the microorganism cell wall.

Preferably the substrate is a stem pentapeptide cell wall precursor, and the peptidyltransferase adds a single glycine to it which can form a cross-bridge through D-ala transpeptidation.

In preferred embodiments the first peptidyltransferase acts on a stem pentapeptide substrate which terminates D-ala-D-ala e.g. the FemX polypeptide as described herein.

Regarding the second peptidyltransferase, this may likewise add a single glycine to a stem pentapeptide substrate which can form a cross-bridge through D-ala transpeptidation.

In one embodiment the presence of the antibiotic in the sample may induce additional enzymes which modify stem pentapeptide cell wall precursors such as to provide a substrate for the second peptidyltransferase. The additional enzymes may be present in the same genomic cluster as the second peptidyltransferase. They may, for example, be the the VanHAX enzymes as described herein which modify the stem pentapeptide substrate from a terminal D-ala-D-ala to a terminal D-ala-D-lac which can be acted on by the VanF polypeptide.

However where the second peptidyltransferase is capable of using the same substrate as the first peptidyltransferase, then there is no need to ensure that additional enzymes are present (whether endogenously or otherwise) or induced.

In another aspect of the invention there is provided a process of producing a microorganism, which may be used in any of the methods of the invention disclosed herein e.g. detection of antibiotics, which process comprises inactivating in the microorganism a first endogenous gene encoding peptidyltransferase activity,
wherein said activity is necessary for growth of the microorganism,
and wherein said activity can be substituted by a second, different, peptidyltransferase, which second peptidyltransferase is inducible in the microorganism by the presence of an antibiotic.

“Inactivation” in the various aspects of the invention includes disruption by any means e.g. deleting all or part of the gene, or introducing a lesion therein.

Generally speaking, those skilled in the art are well able to construct vectors and design protocols for gene inactivation, for example by mutagenesis. For further details see, for example, Molecular Cloning: a Laboratory Manual: 2nd edition, Sambrook et al, 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

Any of the various preferred embodiments of the invention discussed in relation to the first aspect of the invention are applicable here also.

For embodiments in which the bacterium is a Streptomyces strain, reference is also made to Hopwood, D. A., Bibb, M. J., Chater, K. F., Kieser, T., Bruton, C. J., Kieser, H. M., Lydiate, D. J., Smith, C. P., Ward, J. M., and Schrempf, H. (1985) “Genetic Manipulation of Streptomyces: A Laboratory Manual”. Norwich: The John Innes Foundation and Kieser, T., Bibb, M. J., Buttner, M. J., Chater, K. F., and Hopwood, D. A. (2000). “Practical Streptomyces Genetics”. The John Innes Foundation, Norwich.

In a preferred embodiment the first endogenous gene encoding peptidyltransferase activity is inactivated by introducing therein a heterologous marker sequence. This may be achieved by use of flanking sequences which are present in the first peptidyltransferase.

A preferred marker sequence is an apramycin resistance cassette (apr) e.g. isolated from pIJ773, (available from PBL) by EcoRI/HindIII digestion.

The inactivation process may or may not be followed by further manipulations to remove the marker sequence.

In a further aspect there is provided a microorganism, which may be used in any of the methods of the invention disclosed herein e.g. detection of antibiotics,
wherein the microorganism is characterized in that it includes
a first endogenous gene encoding peptidyltransferase activity which is inactivated, which activity is necessary for growth of the microorganism, and which activity can be substituted by
a second, different, peptidyltransferase, which second peptidyltransferase is inducible in the microorganism by the presence of the antibiotic.

In further aspects of the present invention there are provided systems for detecting an activity of an antibiotic in a sample comprising: (a) a mutant microorganism as described above, (b) means for detecting the viability of the microorganism in the presence of the antibiotic.

Also embraced within the scope of the present invention are kits for performing the various aspects of the invention. For instance a kit suitable for use in the first aspect may comprise a preparation of the microorganism, plus further means for carrying out the contact or observation steps e.g. buffers.

Naturally the methods and systems of the invention described above could be used as a primary screen, with further screens (e.g. based on antibiosis of target organisms, which may be different species to the screening microorganism) being employed to further exclude compounds not having the desired activity.

Additionally, the microorganisms of the present invention may, in the presence of appropriate antibiotics which confer viability, be used in test systems for yet further antibiotics—for instance where it is desired to screen for antibiotics acting via different mechanisms to those which induce the second peptidyltransferase.

In another aspect of the invention there is provided a process of producing an isolated antibiotic which affects cell integrity, which method comprises the steps of:
(a) performing a method of the invention as described above such as to identify the activity of the antibiotic in a sample,
(b) isolating the antibiotic from the sample.

Optionally the process is preceded by the step of providing a mutant microorganism as described above.

The invention will now be further described with reference to the following non-limiting Figures and Examples. Other embodiments of the invention will occur to those skilled in the art in the light of these.

### FIGURES

FIG. 1. FemX can add the single gly cross-bridge to stem pentapeptides terminating D-ala-D-ala, but not to stem pentapeptides terminating D-ala-D-lac. VanF can add the single gly cross-bridge to the stem pentapeptides terminating D-ala-D-lac.

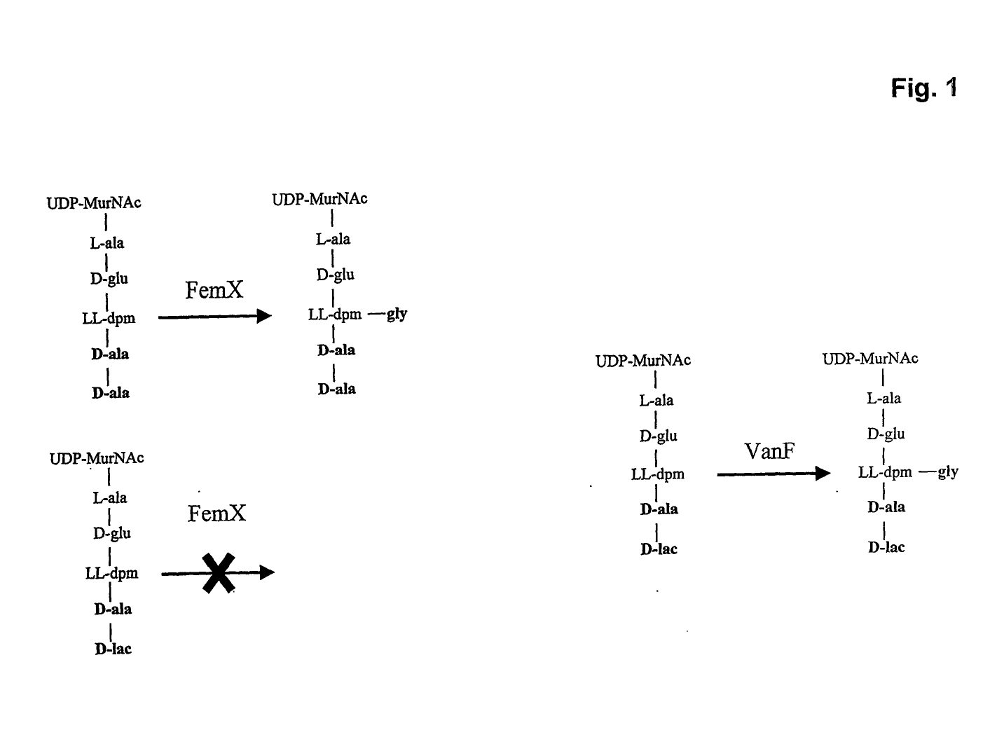

FIG. 2. The enzyme transpeptidase crosslinks adjacent polysaccharide chains through their peptide side chains. The transpeptidase cleaves off the terminal alanine, and joins the remainder to a glycine crossbridge from an adjacent polysaccharide chain.

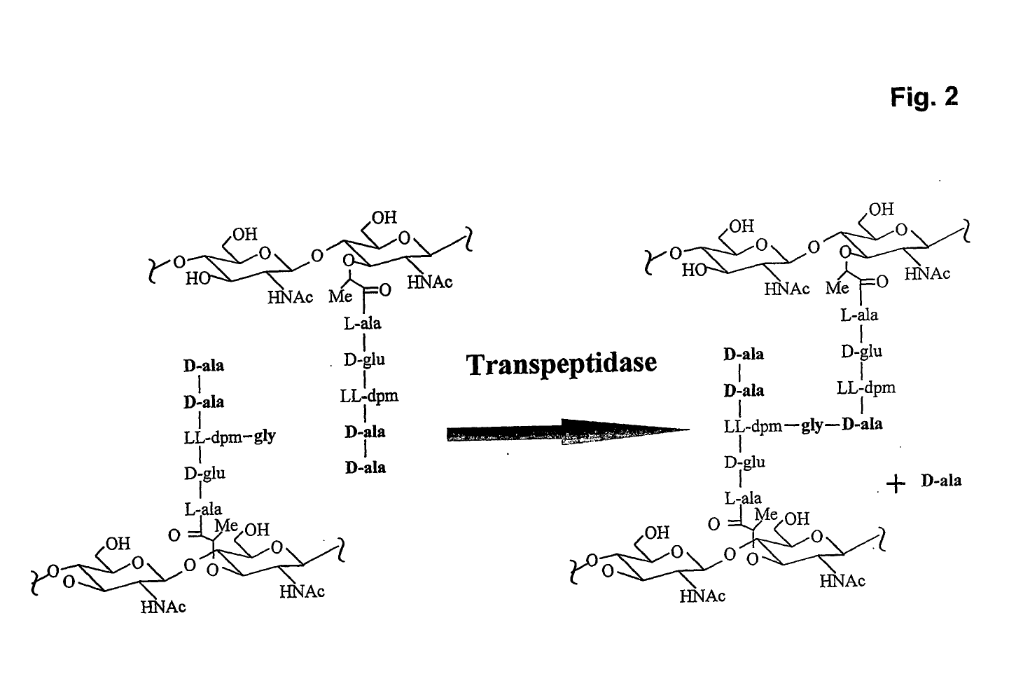

FIG. 3. The enzyme transpeptidase catalyses essentially the same reaction as in FIG. 2 when the cell wall precursors terminate D-ala-D-lac. In this case, the transpeptidase cleaves off the terminal D-lac, and joins the remainder to a glycine crossbridge from an adjacent polysaccharide chain.

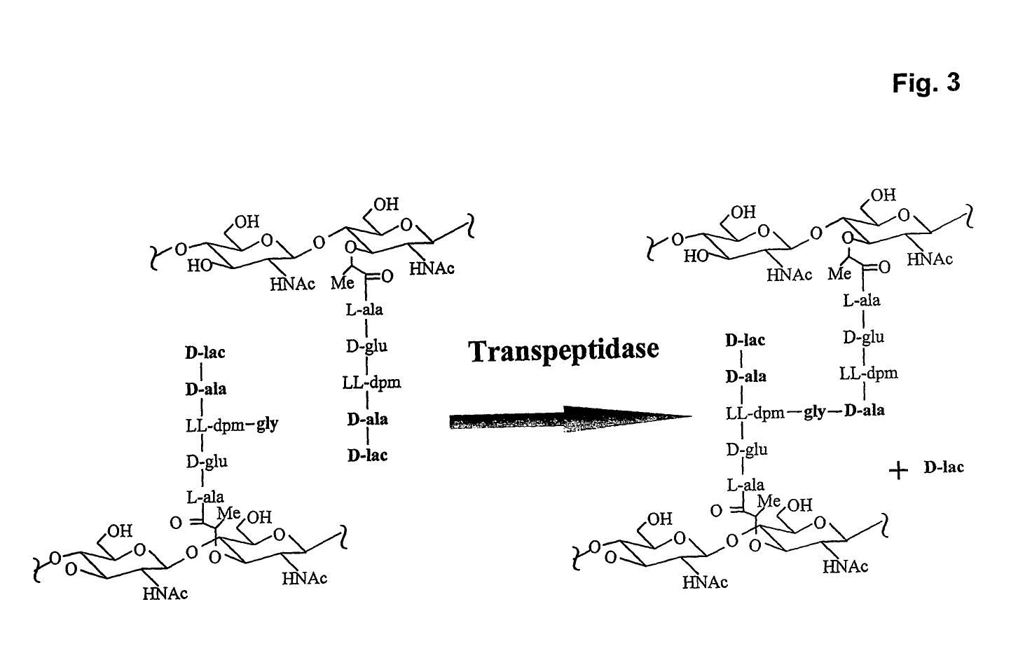

FIG. 4. femX is non-essential provided that vanF is expressed. Therefore, femX null mutants only grow in the presence of vancomycin or other inducers of the van genes, but die in the absence of such inducers. In other words, femX null mutants are drug-dependent strain that can only grow in the presence of van gene inducers.

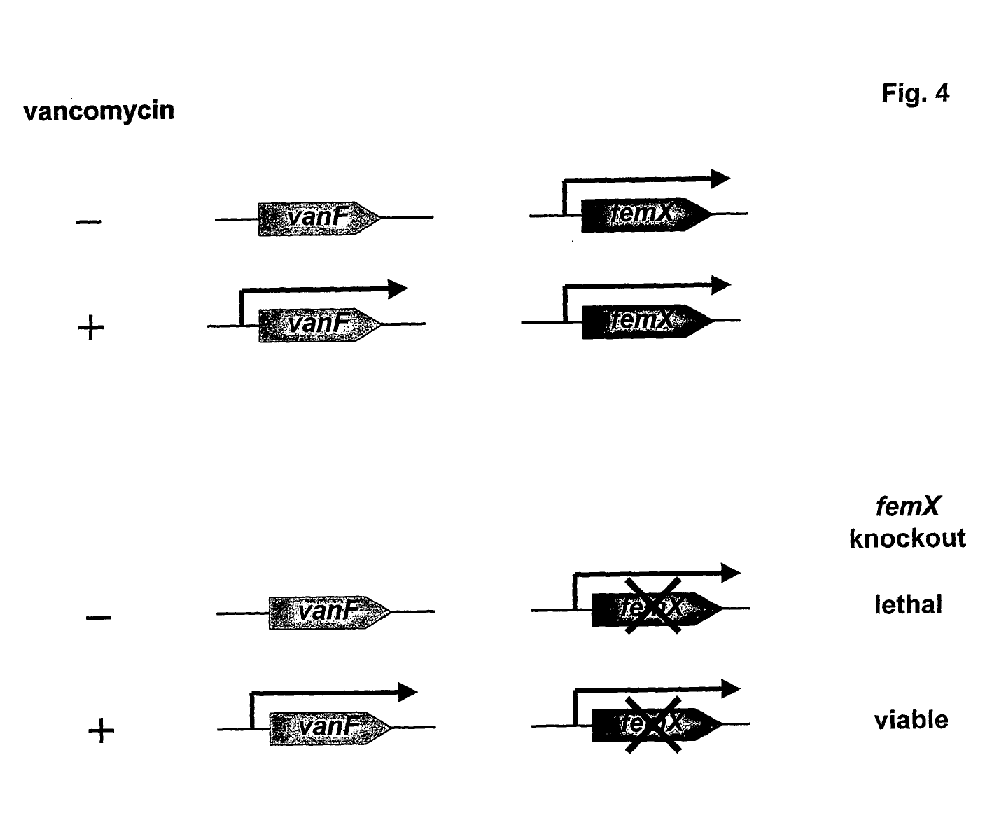

Table 1 summarizes the results of the experiment described in Example 2, in which 13 antibiotics were tested for their effects on the growth of the test strain.

Table 2 shows a sensitivity test using vancomycin.

### EXAMPLES

### Example 1

### Creation of J3130, a ‘Drug-Dependent’ Streptomyces coelicolor Strain Unable to Grow in the Absence of Inducers of the Van Gene Cluster

Replacement of femX (=SC03904) on cosmid H24.

The apramycin resistance cassette (apr) was isolated from pIJ773 (available from PBL) by EcoRI/HindIII digestion, gel purified and then amplified by PCR using the forward femX primer (5′-ACCCATGGGGACACACCAGCCCGAGGAGCGCCCCGAATGATTCCGGGGATCCGTCGACC-3′) and the reverse femX primer (5′-GCTGTCAGAGGTGCGGATCGGGGATGGGCGGTGCGGTCATGTAGGCTGGAGCTGCTTC-3′). The PCR product (the apr cassette with femX flanking DNA) was then gel purified and eluted in 20 μl autoclaved distilled water.

Electrocompetent cells of E. coli strain BW25113 (Datsenko and Wanner, 2000) carrying pIJ790 (available from PBL) and cosmid H24 (Redenbach et al., 1996; available from the John Innes Centre) were prepared as described (Gust et al, 2003) and 50 μl cells were electroporated with 300 ng purified PCR product in a 0.2 cm ice-cold electroporation cuvette using a Bio-Rad gene Pulser II set to 200 Ω, 25 μF and 2.5 Kv. Ice-cold LB (Kieser et al, 2000) (1 ml) was immediately added to shocked cells followed by incubation, shaking, at 37° C. for 1 hour. Cells were harvested by brief centrifugation, resuspended in 100 μl LB and plated onto L-agar plates (Kieser et al, 2000) containing kanamycin (50 μg ml⁻¹)+carbenicillin (100 μg ml⁻¹)+apramycin (50 μg ml⁻¹). After 18 hours growth at 37° C. cosmid DNA was isolated from transformant colonies using alkaline lysis followed by pheol-choloroform extraction (Sambrook et al, 1989) and the femX::apr disruption was confirmed by restriction digestion with BamHI and by PCR using a forward test primer (5′-CCCGAGGAGCGCCCCGAATG-3′) and a reverse test primer (5′-GGGGATGGGCGGTGCGGTCA-3′) which anneal to DNA sequences either side of the disrupted femX gene. The apr cassette contains a unique BamHI site not present in the femX gene such that digestion of wild type cosmid DNA gives a different restriction pattern after separation on an agarose gel compared to cosmid DNA containing the apr disrupted femX gene. In addition the apr cassette differs in size to the femX gene such that PCR using femX flanking primers on wild type and mutated cosmids will give products that are different in size, again analyzed by agarose gel electrophoresis.

Transfer of the Mutagenised Cosmid to S. coelicolor.

Mutagenised cosmid DNA was used to transform the dam, dcm strain ET12567/pUZ8002 (available from the John Innes Centre) by electroporation (as above) and introduced into S. coelicolor strain M600 (available from the John Innes Centre) by conjugation (Gust et al, 2003). Apramycin-resistant (Apr^{R}) exconjugants were selected on SFM medium (Kieser et al, 2000) and screened for kanamycin sensitivity (Gust et al, 2003).

Isolation of Double Crossovers.

Spores isolated from Apr^{R}, kanamycin-resistant (Kan^{R}) colonies were plated onto SFM medium+apramycin (50 μg ml⁻¹)+vancomycin (10 μg ml⁻¹). In the presence of vancomycin, kanamycin-sensitive femX null mutant colonies were readily isolated, and one was designated J3130. However, femX mutants were unable to grow in the absence of vancomycin.

### Example 2

### Testing of the Reporter Strain

To see if growth of J3130 could be induced by control antibiotics known to target the cell envelope, spores of J3130 were spread on MMT medium (Kieser et al, 2000) and potential inducers were applied on paper discs to the freshly spread plates. The results for 16 antibiotics are shown in Table 1. All of the glycopeptides tested (vancomycin, ristocetin, chloroeremomycin and A47934) induced a halo of growth around the paper disc. None of the other antibiotics tested, including many cell wall-specific non-glycopeptide antibiotics, induced growth of J3130.

Table 1 summarizes the results of the assay for the antibiotics used. Thus, it is clear that the bioassay detects a wide variety of glycopeptide antibiotics, thereby allowing the system to act as a generic screen for glycopeptide antibiotics.

These results clearly show the utility of the system as a screen. For instance, an initial assessment of sensitivity demonstrated that 80 ng of vancomycin gives a positive reaction in the bioassay (Table 2).

### Example 3

### Use of Reporter Strain in Bioassay

In order to perform the assay of the invention, spores of J3130 are spread on MMT medium at concentration of approximately 5×10⁶/12 cm² plate. Test compound is applied on paper discs to a number of freshly spread plates in parallel using a different concentration of the test compound in each plate. A halo of growth indicates that the test compound is an inducer of the van genes.

### REFERENCES

Ausubel et al. eds., Current Protocols in Molecular Biology, Second Edition, John Wiley & Sons, 1992.
Datsenko, K. A., and Wanner, B. L. (2000) One-step inactivation of chromosomal genes in Escherichia coli K-12 using PCR products. Proc Natl Acad Sci USA 97: 6640-6645.
Gust, B., Challis, G. L., Fowler, K., Kieser, T. and Chater, K. F. (2003). PCR-targeted Streptomyces gene replacement identifies a protein domain needed for biosynthesis of the sesquiterpene soil odor geosmin. Proc. Natl. Acad. Sci. USA 100: 1541-1546.
Lai, M. H., and Kirsch, D. R. (1996) Induction signals for vancomycin resistance encoded by the vanA gene cluster in Enterococcus faecium. J Bacteriol. 40: 1645-1648.
Redenbach, M, Kieser H M, Denapaite D, Eichner A, Cullum J, Kinashi H, Hopwood D A (1996) A set of ordered cosmids and a detailed genetic and physical map for the 8 Mb Streptomyces coelicolor A3(2) chromosome. Mol Microbiol 21: 77-96
Sambrook, J., Fritsch, E. F. and Maniatis, T. (1989). Molecular Cloning: A Laboratory Manual. Cold Spring Harbor, New York: Cold Spring Harbor Laboratory Press.

Ulijasz, A. T., Grenader, A., and Weisblum, B. (1996) A vancomycin-inducible lacZ reporter system in Bacillus subtilis: induction by antibiotics that inhibit cell wall synthesis and by lysozyme. J Bacteriol 178: 6305-6309.

TABLE 1
13 antibiotics known to target the cell envelope and three antibiotics that do not target
the cell envelope were tested for their ability to induce growth of J3130 in the bioassay
(✓ = induced a halo of growth; x = did not induce a halo of growth).

| | | | | Cephalosporins | | | | Antibiotics that do not | |
|---|---|---|---|---|---|---|---|---|---|
| | | | | and other | | | | target the cell envelope | |
| Glycopeptides | | Penicillins | | β-lactams | | Peptides | | at all | |
| Vancomycin | ✓ | Penicillin G | x | Cefaclor | x | Bacitracin | x | Novobiocin | x |
| | | | | | | | | (target - DNA gyrase) | |
| Ristocetin | ✓ | Penicillin V | x | Cefadroxil | x | | | Thiostrepton | x |
| | | | | | | | | (target - the ribosome) | |
| A47934 | ✓ | Amoxycillin | x | Cephapirin | x | | | Streptomycin | x |
| | | | | | | | | (target - the ribosome) | |
| Chloroeremomycin | ✓ | Ampicillin | x | Cepharadine | x | | | | |

TABLE 2
Two-fold dilution series test of vancomycin from 10
μg on femX mutant (✓ = induced growth;
x = did not induce growth, in the bioassay).

| Amount of vancomycin μg | Growth |
|---|---|
| 10 | ✓ |
| 5 | ✓ |
| 2.5 | ✓ |
| 1.3 | ✓ |
| 0.6 | ✓ |
| 0.3 | ✓ |
| 0.15 | ✓ |
| 0.08 | ✓ |
| 0.04 | x |
| 0.02 | x |
| 0.01 | x |
| 0.005 | x |
| 0.002 | x |
| 0.001 | x |
| 0.0005 | x |
| 0.0003 | x |

1. A method of detecting an activity of an antibiotic in a sample, the method comprising the steps of:
(a) providing a microorganism in which a first endogenous gene encoding peptidyltransferase activity is inactivated, which activity is necessary for growth of the microorganism, and which activity can be complemented by a second, different, peptidyltransferase, which second peptidyltransferase is inducible in the microorganism by the presence of the antibiotic,
(b) contacting the sample with the microorganism, and
(c) observing the microorganism for growth, wherein growth of the microorganism is correlated with the presence of the antibiotic.

2. The method of claim 1 wherein the antibiotic is a glycopeptide antibiotic which interferes with the physical integrity of the cell envelope.

3. The method of claim 1 wherein the second peptidyltransferase is endogenous.

4. The method of claim 1 wherein the peptidyltransferase activity is nonribosomal and operates on a substrate in the cell involved in cross-bridge formation of the microorganism cell wall.

5. The method of claim 4 wherein the peptidyltransferase activity adds a single glycine to a stem pentapeptide substrate which can form a cross-bridge through D-ala transpeptidation.

6. The method of claim 5 wherein the first peptidyltransferase acts on a stem pentapeptide substrate which terminates D-ala-D-ala.

7. The method of claim 6 wherein the first endogenous gene encoding peptidyltransferase activity is femX(SC03904).

8. The method of claims 5 wherein the second peptidyltransferase acts on a stem pentapeptide substrate which terminates D-ala-D-lac.

9. The method of claim 8 wherein the second peptidyltransferase is encoded by vanF (SC03593).

10. The method of claims 5 wherein the presence of the antibiotic in the sample induces additional enzymes which modify stem pentapeptide cell wall precursors such as to provide a substrate for the second peptidyltransferase.

11. The method of claim 10 wherein the additional enzymes may be present in the same genomic cluster as the second peptidyltransferase.

12. The method of claim 10 wherein the additional enzymes are vanHAX enzymes encoded by vanH (SC03594); vanA (SC03595); or vanX (SC03596).

13. The method of claim 1 wherein the bacterium is an actinomycete.

14. The method of claim 13 wherein the bacterium is Streptomyces.

15. The method of claim 14 wherein the bacterium is Streptomyces coelicolor.

16. The method of claim 15 wherein the bacterium is Streptomyces coelicolor A3 (2).

17. The method of claims 2 wherein the microorganism is a strain in which enzymes which may otherwise degrade glycopeptidic antibiotics have been inactivated.

18.-20. (canceled)

21. A process of producing a microorganism for use in the method of claim 1, which process comprises inactivating in the microorganism a first endogenous gene encoding peptidyltransferase activity, wherein said activity is necessary for growth of the microorganism, and wherein said activity can be substituted by a second, different, peptidyltransferase, which second peptidyltransferase is inducible in the microorganism by the presence of an antibiotic.

22. The process of claim 21 wherein the first endogenous gene encoding peptidyltransferase activity is inactivated by introducing therein a heterologous market sequence.

23. The process of claim 21 wherein the second peptidyltransferase is endogenous.

24. The process of claim 21 wherein the microorganism is transformed with a gene encoding the second peptidyltransferase.

25. A process of producing an isolated antibiotic which affects cell integrity, which method comprises the steps of:
(a) performing a method according to claims 1 such as to identify the activity of the antibiotic in a sample, and
(b) isolating the antibiotic form the sample.

26. The process of claim 25 which is preceded by the step of providing a transformed microorganism according to the process claim 21.

27. A microorganism for use in the method of claim 1, which microorganism is characterized in that it includes a first endogenous gene encoding peptidyltransferase activity which is inactivated, which activity is necessary for growth of the microorganism, and which activity can be substituted by a second, different, peptidyltransferase, which second peptidyltransferase is inducible in the microorganism by the presence of the antibiotic.

28. A system for detecting an activity of an antibiotic in a sample comprising:
(a) the transformed microorganism of claim 27, and
(b) means for detecting the viability of the microorganism in the presence of the antibiotic.

29. A kit for performing the method according to claim 1, wherein the kit comprises a preparation of the microorganism, plus a further means for carrying out the contact or observation steps.
